(19) United States
(12) Patent Application Publication
(10) Pub. No.: US 2022/0126040 A1
(43) Pub. Date: Apr. 28, 2022

(54) APPARATUS FOR DELIVERY OF A FLUID MEDICAL GAS TO PATIENTS

(71) Applicant: Modem Nose Clinic LLC, Salem, OR (US)

(72) Inventor: Nahmjee Lee Skarada, Salem, OR (US)

(73) Assignee: Modern Nose Clinic LLC, Salem, OR (US)

(21) Appl. No.: 17/429,213

(22) PCT Filed: Feb. 7, 2019
(86) PCT No.: PCT/US19/17121

§ 371 Date: Aug. 6, 2021

(51) Int. Cl. A61M 16/01; A61M 16/06; A61M 16/04; A61M 16/20

(52) U.S. Cl. CPC A61M 16/01; A61M 16/0605; A61M 2210/0625; A61M 16/208; A61M 16/0495

(57) ABSTRACT

Apparatus for direct oral delivery of fluid medical gas to a patient, comprising: a conduit, a mouthpiece in fluid communication with the conduit, an exhalant chamber at least partially divided from the conduit, an outlet valve in one-way fluid communication between the conduit and the exhalant chamber, the outlet valve adapted for permitting exhalation of exhalant by the patient into the exhalant chamber and adapted for porting of the exhalant outside of an environment in which the apparatus is adapted for use.

### TECHNICAL FIELD

The present invention relates in general to devices used in the process of sedation of a patient for performing surgery and other procedures on the patient, and more particularly to an apparatus for delivery of fluid medical gases, such as mixed oxygen and Nitrous Oxide (N₂O), for sedation able to be administered to a patient to more readily enable performance of such surgery and other procedures on an out-patient clinic-type, or surgi-center-type, basis.

### BACKGROUND ART

Fluid medical gas combinations, such as Nitrous Oxide (N₂O) mixed with Oxygen (O₂), have long been used in oral surgery operatories and dental offices to manage patient anxiety and pain during oral surgery and other dental procedures. As such, medical gas combinations have been commonly used as anesthetics in conjunction with local anesthetics to numb areas of the mouth where such surgery and procedures have been performed. As used herein, fluid medical gas typically comprises combined gases, whether with ambient air or diverse medical gas supplies such as, for example, N₂O and O₂.

N₂O mixed with O₂ has been traditionally delivered using devices adapted for introducing a mixture of the O₂ and the N₂O into the lungs via the patient's nostrils and airways where the N₂O can be most effectively introduced into the bloodstream of the patient to produce desired anxiolytic properties. However, such traditional nasal delivery devices, an exemplary one of which is shown in FIGS. 1A and 1B, have not been well-suited for performing nasal surgery and procedures, and indeed have not been traditionally used for such, since such traditional devices have included a nasal canula and vacuum assist scavenger mask that would have blocked the easiest and most direct access by doctors, through the nostrils, to the very nasal regions to be treated.

Accordingly, other methods and devices for sedation have been utilized, including the use of intravenous administration and endotracheal tube (ET) of traditional general anesthesia, using drugs such as Propofol, Isoflurance, and Sevoflurance, when performing nasal surgery and procedures in the past, and this has contributed to unnecessarily high costs and increased recovery times for patients receiving such procedures performed in hospitals or in-patient surgical centers requiring additional anesthesiologists, increased nursing care, and correspondingly larger operatories, recovery facilities, and administrative overhead.

With more traditional anesthetics, patient's risks, such as bleeding, prolonged comatose state, or even death, are higher. Further patient recovery time is slower, such that quicker return to normal activities including driving an automobile, returning to work, etc., are delayed. And while driving a vehicle immediately following any surgery or procedure is never recommended, patient and related family member experiences involving methods of conscious sedation, such as with N₂O, have been rated superior in terms of overall patient well-being, both during and after the performance of surgery and procedures.

Accordingly, there has been needed a device and method of allowing administration of medical gas combinations under proper supervision of trained medical professionals for relief of pain and anxiety associated with nasal surgery and other procedures involving the nose or nasal passages, or any other surgery or procedure where traditional devices and methods for delivery of medical gases has not been otherwise feasible, suitable, or advantageous because such has involved blocking access to areas to be treated.

### SUMMARY OF THE INVENTION

In accordance with an aspect of the disclosure, there is provided an apparatus for oral delivery (preferably exclusively and directly through the mouth) of a fluid medical gas to a patient. The apparatus comprises: a conduit adapted for fluid communication with a fluid medical gas supply for introduction of the fluid medical gas into the patient's mouth, a mouthpiece in fluid communication with the conduit and adapted for facilitating the introduction of fluid medical gas directly into the patient's mouth, and an exhalant chamber at least partially divided from the conduit.

There is preferably provided at least one inlet port, but in an embodiment in accordance with an aspect of the disclosure preferably a plurality of inlet ports (e.g., preferably two inlet ports) preferably comprising tubes passing through a wall of, and into communication with, the conduit, rendering it adapted for fluid communication between the conduit and the fluid medical gas supply for delivery of the fluid medical gas into the patient's mouth through the conduit. Such tubes passing through the wall of the conduit each have openings adapted for sealed interconnection with available medical gas supply tubes, and thus the inlet ports of the apparatus are adapted for receiving fluid medical gas from typically available medical gas supply lines in a suite available for providing surgery, treatment and procedures.

The apparatus is further comprised of an outlet valve in one-way fluid communication between the conduit and the exhalant chamber, the outlet valve adapted for permitting exhalation of combined breath, exhaled fluid medical gas, and any saliva, as exhalant by the patient, into the exhalant chamber. Further, the outlet valve prevents inhalation of any fluid gas present in the exhalant chamber, wherein the exhalant chamber is adapted for porting of the exhalant outside of an environment in which the apparatus is adapted for use, for example through supplied evacuation tubing leading to an outside, open-air, location.

Preferably the mouthpiece further comprises an outer lip adapted for sealing against the patient's lips to facilitate delivery of fluid medical gas into the patient's mouth, the mouthpiece also preferably further comprises a bite member extending integrally from the mouthpiece lip member so as to be adapted for insertion between the patient's teeth and to facilitate the patient's ability to hold the mouthpiece in the patient's mouth, and the mouthpiece preferably surrounds the conduit, for facilitating delivery of the fluid medical gas into the patient's mouth.

In an embodiment in accordance with an aspect of the disclosure there is comprised a dividing wall defining the conduit as separate from the exhalant chamber, wherein the conduit is defined in integral communication with and posterior of the mouthpiece. In this embodiment, the exhalant chamber further comprises an outer concave chamber wall anterior of and partially surrounding the conduit and mouthpiece combination, the exhalant chamber having spaced ribs (on either the outer exhalant chamber wall or the inner exhalant chamber wall) for forming a vented cavity (which is the exhalant chamber) for scavenging exhalant and positioned between the outer chamber wall and the conduit and mouthpiece combination, and wherein said conduit and mouthpiece combination are at least partially divided from the exhalant chamber by the dividing wall defining the conduit as separate from the exhalant chamber.

Preferably, in an embodiment in accordance with an aspect of the disclosure, the conduit, the mouthpiece comprise an integrally molded unit, while preferably further the dividing wall is also part of an integrally molded unit together with such an integral conduit and mouthpiece. Preferably the integral mouthpiece, conduit, and dividing wall are all made of a plastic material, such as Thermoplastic elastomer (TPE) or soft silicone material. Moreover, preferably in accordance with such an embodiment the exhalant chamber partially surrounds the integral unit comprised of the conduit, the mouthpiece, and the dividing wall, wherein the exhalant chamber preferably comprises an integral unit defining at least one outlet port, but preferably a plurality (e.g., two) outlet ports, adapted for fluid communication with, and interconnection to, tubing connected to the vacuum system for evacuating the exhalant to an environment outside of the room in which the surgery or procedure is being performed.

In an alternative embodiment in accordance with another aspect of the disclosure, there is provided an apparatus for oral delivery of fluid medical gas to a patient in conjunction with a nasal occlusal device 71, 73. This device can be inserted to the posterior aspect of a patient's nasal cavity toward the nasopharyngeal junction to prevent transmission of fluids from nasal cavity into oral cavity during performance of surgery or a procedure while using the apparatus. Such a nasal occlusal device can be single use disposable (such as gauze on a tether) or reusable (such as a balloon-type catheter).

In an embodiment of the invention, there is provided an apparatus for oral delivery of fluid medical gas to a patient, comprising: a mouthpiece attached to, or interconnected with, and preferably integrated with, a conduit posterior of the mouthpiece, the conduit defining a channel with openings at least one of which is aligned with the opening defined in the mouthpiece so as to be adapted for fluid communication with fluid medical gas supply for introduction of the fluid medical gas directly into the patient's mouth. The conduit further defines at least one inlet port, but preferably a plurality of inlet ports (e.g., two inlet ports), adapted for fluid communication between the conduit opening, the mouthpiece opening, and the fluid medical gas supply.

The apparatus in accordance with this embodiment of the disclosure further comprises: an inner exhalant chamber wall molded at least partially around the mouthpiece so that fluid medical gas passing through the conduit and the opening in the lip of the mouthpiece is prevented from flowing directly into the exhalant chamber, the exhalant chamber further comprising at least one outlet port, but preferably a plurality (e.g., two) of outlet ports adapted for fluid communication with tubing connected to a vacuum system for evacuating the exhalant.

Preferably, each of the plurality of inlet ports communicates in sealed fashion through the exhalant chamber and the mouthpiece and opens into the conduit adapted for allowing flow of fluid medical gas from the fluid medical gas supply into the conduit, and wherein each of the plurality of outlet ports communicates through the exhalant chamber wall to open into the exhalant chamber adapted for allowing evacuation of exhalant through the tubing under vacuum pressure.

The apparatus in accordance with this embodiment of the disclosure further comprises an outlet valve in one-way fluid communication between the conduit and the exhalant chamber, the outlet valve adapted for permitting exhalation of combined breath, exhaled fluid medical gas, and any saliva, as exhalant by the patient through the conduit and into the exhalant chamber. Further, the outlet valve prevents inhalation of any fluid gas present in the exhalant chamber, the exhalant chamber being adapted for porting of the exhalant preferably through a plurality of tubes interconnected with the plurality of outlet ports for porting exhalant outside of the environment in which the apparatus is adapted for use.

Preferably the mouthpiece in accordance with this embodiment further comprises an outer lip adapted for sealing against the patient's lips to facilitate delivery of fluid medical gas into the patient's mouth, the mouthpiece also preferably further comprises a bite member extending integrally from the mouthpiece lip member so as to be adapted for insertion between the patient's teeth and to facilitate the patient's ability to hold the mouthpiece in the patient's mouth, and the mouthpiece preferably surrounds the conduit, for facilitating delivery of the fluid medical gas into the patient's mouth.

In accordance with any foregoing aspect or embodiment of the disclosure, there may be provided an alternative embodiment wherein the mouthpiece of the apparatus further comprises a combination bite member and tongue engagement member adapted for insertion between the patient's upper and lower teeth and engagement in depressing the patient's tongue to facilitate the patient's ability to hold the mouthpiece in the patient's mouth for delivery of the fluid medical gas into the patient's mouth.

In an embodiment there is provided an apparatus for oral delivery of fluid medical gases to a patient, comprising: a molded inner mouthpiece comprising a annular lip defining an opening, a conduit wall extending from the mouthpiece adjacent the opening in the annular lip of the mouthpiece, the conduit wall defining a channel having first and second end openings, the first end opening communicating with the opening in the mouthpiece.

There are further provided, in accordance with this embodiment, a plurality of laterally-disposed inlet ports communicating through the conduit wall and opening into the channel adapted for conveying fluid medical gas from a fluid medical gas supply directly into a patient's mouth, the opening in the mouthpiece and the channel adapted to allow inhalation suction by the patient of the fluid medical gas.

In accordance with this embodiment there is further provided an inner exhalant chamber wall molded partially around the mouthpiece and the conduit wall, the inner exhalant chamber wall defining an opening therein communicating with the second opening, and hence the channel, of the conduit wall. Still further, there is provided an outer exhalant chamber wall molded partially around the inner exhalant chamber wall and defining a vented exhalant chamber between the outer exhalant chamber wall and the inner exhalant chamber wall, the outer exhalant chamber wall further defining a plurality of laterally-disposed outlet ports adapted for communicating through the outer exhalant chamber wall and thus between the vented exhalant chamber and tubes of a vacuum system for evacuating exhalant outside of the environment in which the apparatus is to be used.

In this embodiment, the inner exhalant chamber wall defines a plurality of laterally-disposed inlet ports adapted from communicating through the outer exhalant chamber wall, through the inner exhalant chamber wall, and through an opening into the conduit wall adapted for communicating with the channel for exclusive oral delivery of medical gas to the patient.

This embodiment of the apparatus further comprises a positive-pressure outlet valve in one-way fluid communication between the channel defined in the conduit wall and the vented exhalant chamber, the outlet valve residing in the opening defined in the inner exhalant chamber wall and adapted for permitting exhalation of combined breath, exhaled fluid medical gas, and any saliva, as exhalant by the patient, into the vented exhalant chamber. Further, the outlet valve prevents inhalation of any fluid gas or saliva present in the vented exhalant chamber. Still further, the vented exhalant chamber is adapted for porting of the exhalant through the plurality of outlet ports to outside of the environment in which the apparatus is adapted for use.

Preferably, in this embodiment of the invention, the outer exhalant chamber wall of the apparatus is integrally molded of plastic or silicone, wherein the inner exhalant chamber wall, the conduit wall, and the mouthpiece are integrally molded of plastic or silicone. Further, preferably, the outer exhalant chamber wall and the inner exhalant chamber wall define outer and inner nested cups, and the outer exhalant wall further comprises a plurality of ribbed spacers for maintaining venting of the exhalant chamber between the outer exhalant chamber wall and the inner exhalant chamber wall.

Still further, preferably the plurality of laterally-disposed inlet ports are comprised of a plurality of tubes passing through the outer exhalant chamber wall, the inner exhalant chamber wall, and through and opening into the conduit wall, the plurality of tubes adapted for interconnecting with corresponding medical gas supply tubes from a medical gas supply cannister. Further, the laterally-disposed outlet ports are comprised of another plurality of tubes passing through the outer exhalant chamber wall and opening into the exhalant chamber, the another plurality of tubes being adapted for interconnecting with corresponding evacuation tubes for scavenging exhalant, together with ambient air through the venting for the exhalant chamber, to the area outside the environment in which the apparatus is to be used.

The embodiments of the apparatus of the present disclosure address the shortcomings of prior art devices and enable a method of administering medical gases orally that has heretofore not been made available in the field of surgery, treatments, and procedures relating to nasal cavities and members, and thus the embodiments of the apparatus of the present disclosure facilitate nose-related treatments, procedures and surgeries such that the same may improve patient experiences and recovery times. Whereas with more traditional anesthetics, patient recovery time has been slower, the apparatus of the present disclosure facilitates quicker returns to normal activities including driving an automobile, returning to work, etc. And while driving a vehicle immediately following any surgery or procedure is never recommended, patient and related family member experiences involving methods of conscious sedation, such as with N₂O are facilitated and more highly-rated in terms of overall patient well-being, both during and after the performance of surgery and procedures.

Accordingly, since there is now provided an apparatus in accordance with one or more embodiments of the disclosure allowing administration of medical gas combinations orally under proper supervision of trained medical professionals for relief of pain and anxiety associated with procedures for which such delivery of medical gases has not been previously feasible, there is now therefore provided an opportunity for achieving the benefits of conscious sedation in such treatments not previously experienced.

The subject matter of the present invention is particularly pointed out and distinctly claimed in the concluding portion of this specification. However, both the organization and method of operation, together with further advantages and objects thereof, may best be understood by reference to the following descriptions taken in connection with accompanying drawings wherein like reference characters refer to like elements.

### BRIEF DESCRIPTION OF THE DRAWINGS

FIG. 1A is a front perspective view of a prior art device for delivery of medical gases via a patient's nostrils;

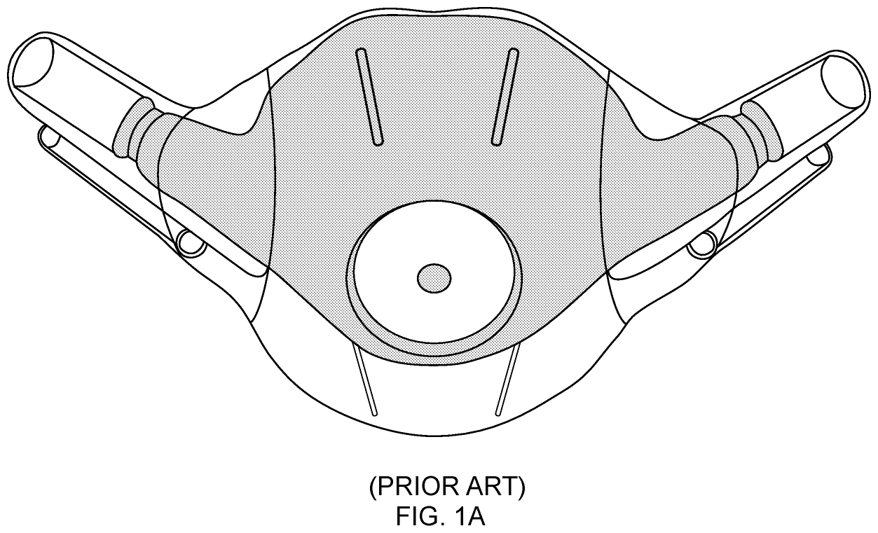

FIG. 1B is a rear perspective view of the prior art device of FIG. 1A for delivery of medical gases via the patient's nostrils;

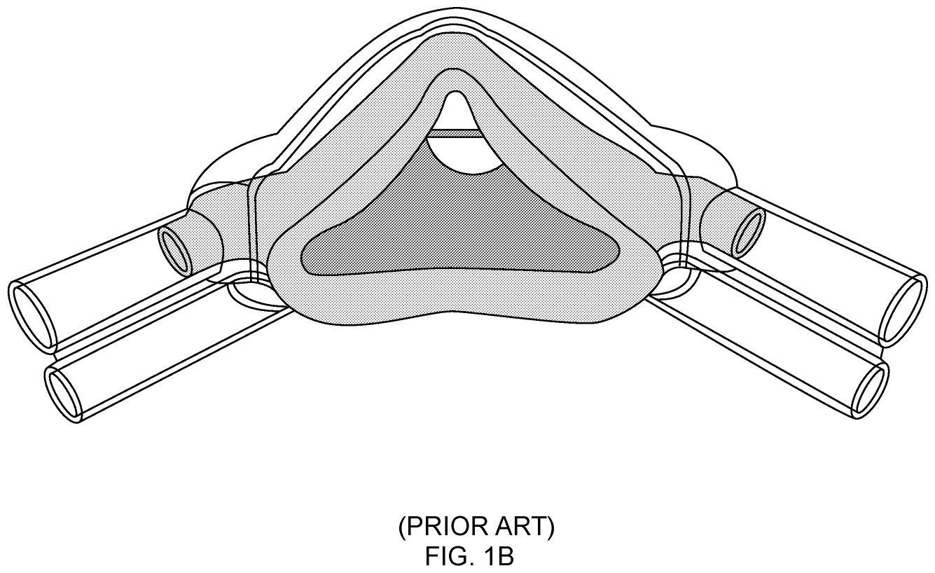

FIG. 2A is a front perspective view of an apparatus for delivery of medical gases via a patient's mouth in accordance with an aspect of the disclosure;

FIG. 2B is a rear perspective view of the apparatus of FIG. 2A for delivery of medical gases via the patient's mouth in accordance with an aspect of the disclosure;

FIG. 2C is a top perspective view of the apparatus of FIG. 2A for delivery of medical gases via the patient's mouth in accordance with an aspect of the invention;

FIG. 3A is a rear perspective view of an alternate embodiment mouthpiece portion of an apparatus for delivery of medical gases via a patient's mouth in accordance with an aspect of the disclosure;

FIG. 3B is a rear perspective view of another alternative embodiment mouthpiece portion of an apparatus for delivery of medical gases via a patient's mouth in accordance with an aspect of the disclosure; and

FIG. 4 is a perspective view of a patient wearing an apparatus for delivery of medical gases via the patient's mouth in accordance with an aspect of the disclosure, and wherein the patient also has installed a device for occluding the rear of the patient's nasal passages at the posterior aspect of nasal cavity near the nasopharyngeal junction to prevent transmission of fluids from the nasal passages into the oral cavity. This nasal occlusion device can be single use gauze, foam or cotton that is shaped or an inflatable balloon type of device.

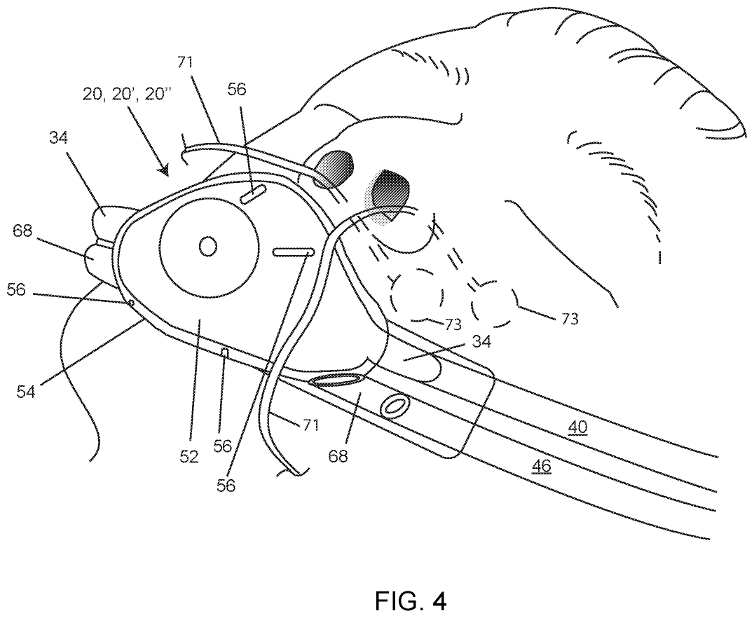

### DETAILED DESCRIPTION

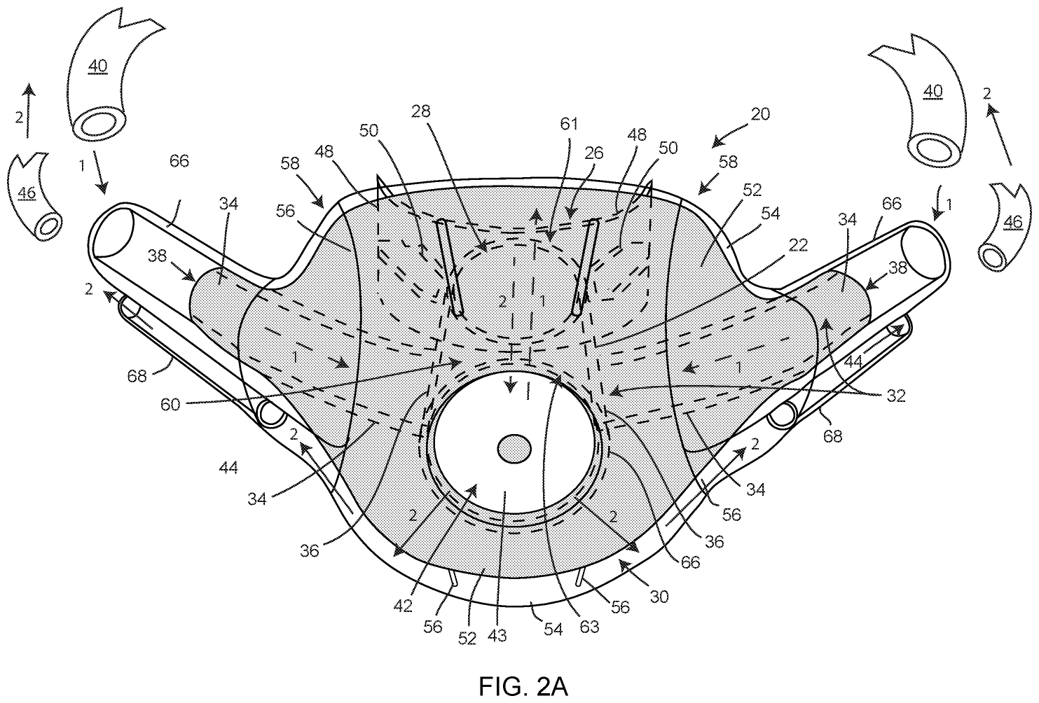

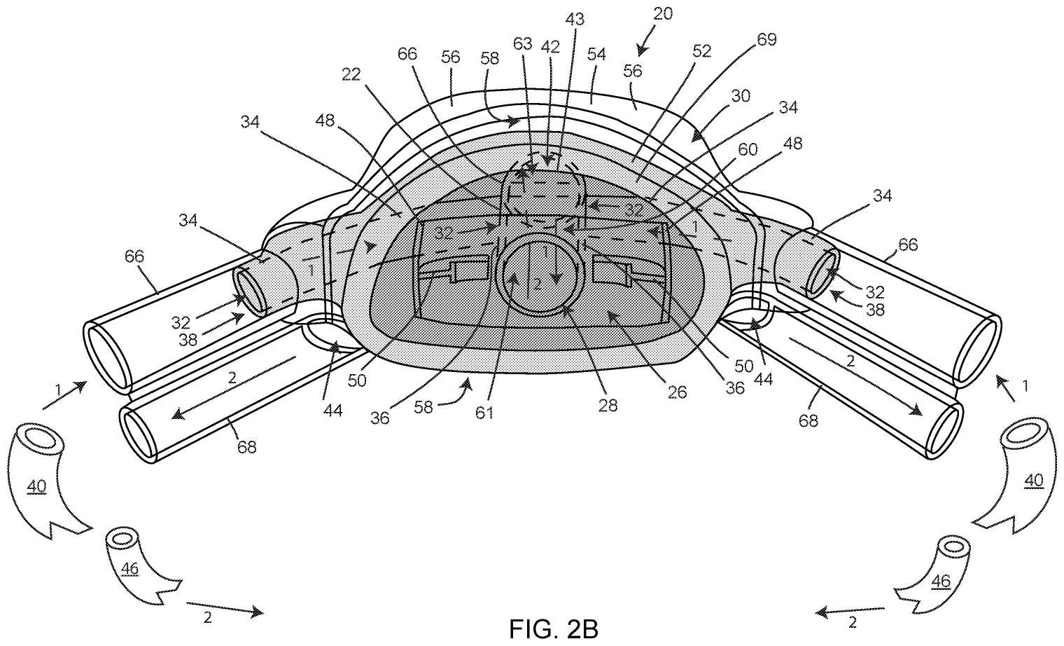

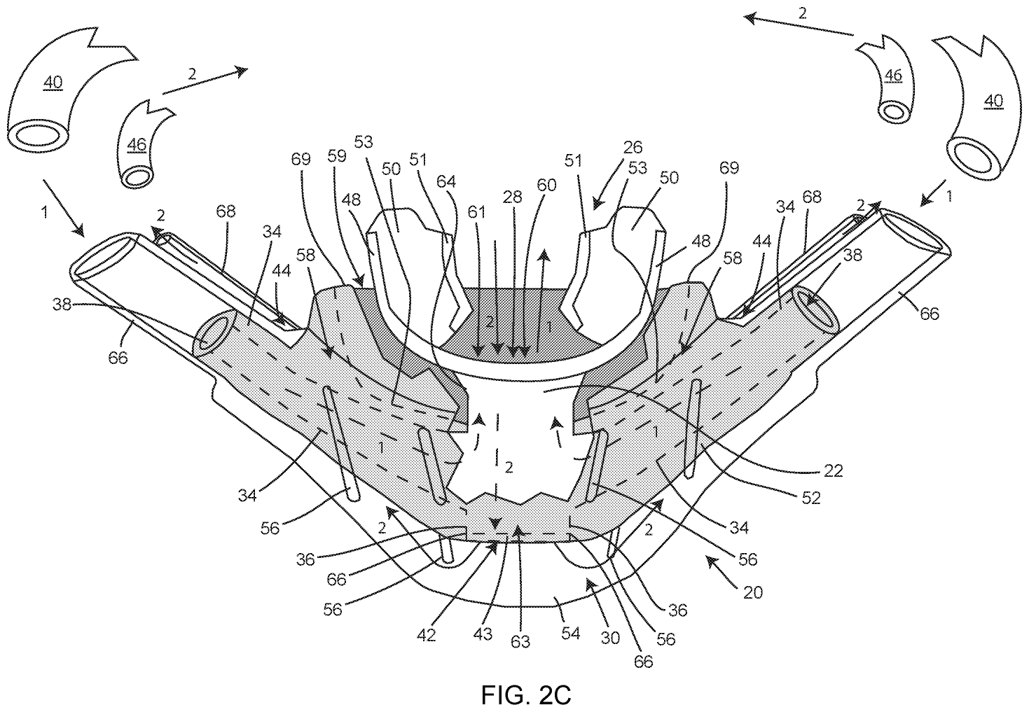

Referring to FIGS. 2A-C and 4, there is provided an apparatus 20 for oral delivery (preferably exclusively and directly through the mouth) of a fluid medical gas to a patient. The apparatus 20 comprises: a conduit 22 comprised preferably of a generally cylindrical conduit wall 36 defining a channel 60 having first and second openings, 61, 66 adapted for fluid communication with a fluid medical gas supply for introduction of the fluid medical gas into the patient's mouth, a mouthpiece 26 defining an opening 28 in fluid communication with the channel 60 of the conduit 22 and adapted for facilitating the introduction of fluid medical gas directly into the patient's mouth, and an exhalant chamber 30 at least partially divided from the conduit. Preferably the channel 60 of the conduit 22 communicates with the opening 28, and preferably the mouthpiece 26 is integral with the conduit, with the conduit being positioned anteriorly of the mouthpiece and so that the opening in the mouthpiece coincides and aligns with the channel of the conduit so as to be adapted for exclusive fluid oral communication between the patient and the fluid medical gas supply provided to the surgery/procedure suite/area through tubing 40.

There is preferably provided at least one inlet port 32 in an embodiment in accordance with an aspect of the disclosure, but preferably a plurality of laterally-disposed inlet ports (e.g., preferably two inlet ports) 32, preferably comprising tubes 34 communicating through the wall 36 of, and into fluid communication with, the channel 60 of the conduit 22, rendering the apparatus 20 adapted for fluid communication between the conduit and the fluid medical gas supply for delivery of the fluid medical gas into the patient's mouth through the conduit.

Thus, tubes 34 are also preferably laterally-disposed and in fluid communication with the channel 60 of the conduit 22, and each of the tubes 34 have openings 38 adapted for sealed interconnection with typically available medical gas supply tubes 40. Thus, the inlet ports 32 of the apparatus 20 are adapted for receiving fluid medical gas from gas supply lines 40 that are available in a room for providing surgery, treatment and procedures on the nose and portions of the nose of a patient.

There is provided a preferably cup-shaped outer exhalant chamber wall 54 which opens posteriorly (i.e., adapted to open toward the patient's face) and a partially-nesting, cup-shaped inner exhalant chamber wall 52 having an inner support structure 53 (referenced in FIG. 2B) and defining a frontal lip opening 59 defined by a rim of the inner exhalant chamber wall and serving as a face engagement member 69. Outer exhalant chamber wall 54 is shown as clear plastic allowing a view within to see shaded inner exhalant chamber dividing wall 52. In an embodiment there is provided an inner structure 53 within and extending from inner surfaces of inner exhalant chamber wall 52, such structure preferably being molded integrally with the inner exhalant chamber wall for supporting the mouthpiece 26 via conduit 22 and tubes 34. Inner exhalant chamber wall 52 defines at its most anterior extent an exhalant opening 42.

The plurality of laterally-disposed tubes 34 providing for inlet of medical gas pass through the outer exhalant chamber wall 54, the inner exhalant chamber wall 52, supported by the inner support structure 53, and through the conduit wall 36 to open into the channel 60. The plurality of tubes 34 are adapted for interconnecting with corresponding medical gas supply tubes 40 from a medical gas supply.

Still further, the laterally-disposed outlet ports 44 are comprised of another plurality of tubes 68 opening into the exhalant chamber 30, pass through the outer exhalant chamber wall 54 where they are adapted for interconnecting with corresponding evacuation tubes 46 for scavenging exhalant, together with ambient air, through the venting for the exhalant chamber, to the area outside the environment in which the apparatus is to be used.

At a second end 37 of the conduit 22, the apparatus 20 is further comprised of a preferably positive pressure outlet valve 43 in one-way fluid communication between the second opening 66 of the channel 60 of the conduit 22 communicating with the opening 42 in the inner exhalant chamber wall 52 into the exhalant chamber 30, the outlet valve adapted for permitting exhalation of combined breath, exhaled fluid medical gas, and any saliva, as exhalant by the patient, into the exhalant chamber 30.

Thus, the outlet valve 43 residing in the opening 42 defined in the inner exhalant chamber wall 52 is adapted for permitting exhalation of combined breath, exhaled fluid medical gas, and any saliva, as exhalant by the patient, into the vented exhalant chamber 30. Further, the outlet valve 43 prevents inhalation of any fluid gas or saliva present in the vented exhalant chamber 30. Still further, the vented exhalant chamber 30 is adapted for porting of the exhalant through the plurality of tubes 68 interconnecting with the plurality of outlet ports 44 for porting exhalant outside of the environment in which the apparatus 20 is adapted for use.

The exhalant chamber 30 is preferably vented between the cup-shaped outer exhalant chamber wall 54 and the inner cup-shaped dividing wall 52, and is adapted for porting, via preferably a plurality of laterally-disposed outlet tubes 68 and ports 44, exhalant along the pathway designated by exhalant pathway arrows 2. Thus, the exhalant is ported to outside of an environment in which the apparatus 20 is adapted for use, for example through supplied evacuation tubing 46 under vacuum pressure and leading to outside the surgery/procedure suite in a manner known in the art of scavenging and properly disposing of medical gasses and exhalant.

In an embodiment, the inner dividing wall 52 and support structure 53 separates the conduit 22, the tubes 34, and the inlet ports 38 as separate from the exhalant chamber 30. Further, within support structure 53, the conduit 22 is defined in integral communication with the opening 28 and is located posterior of the mouthpiece 26. Thus, the combination of the dividing wall 52, the support structure 53, the conduit wall 36 forming the conduit 22 and channel 60, and the tubes 34 forming the inlet ports 38, may all be integrally molded as a single unit even preferably with the mouthpiece 26, all for defining an integral pathway, shown as pathway 1, for oral delivery of fluid medical gas to the patient.

The outer concave exhalant chamber wall 54 partially surrounds the inner exhalant chamber dividing wall 52, which in turn partially surrounds the preferably-integrated mouthpiece 26, the conduit 22, the support structure 53, and the tubes 34, in combination. The exhalant chamber 30 is maintained in an open condition relative to the inner dividing wall 52 and the outer exhalant chamber wall 54 with spaced ribs 56 for maintaining a cavity (which is the exhalant chamber 30) which is vented as shown at arrows 58 with ambient air passing through the spaced openings created by the ribs and under vacuum pressure from the scavenging tubes 46 in the suite for scavenging exhalant. The inner dividing wall 52 is thus positioned between the outer chamber wall 54 and the conduit 22, the support structure 53, the mouthpiece 26, and tubes 34 combination such that the conduit 22, mouthpiece 26, and tubes 34 combination are at least partially divided from the exhalant chamber 30 by the dividing wall 52. The support structure may be comprised of a filler material for lending support for the tubes 34 within the inner dividing wall 52.

Preferably, the inner dividing wall 52 and the outer chamber wall 54 interface, and basically interconnect at the location of the inlet ports 38 defined by the tubes 34, in that the outer chamber wall 54 narrows into inlet tube fittings 67 which, together with the ribs 56, anchor the inner dividing wall 52 combination (together with the mouthpiece 26, conduit wall 36, and tubes 34) within and relative to the outer chamber wall 54 to form and maintain the exhalant chamber 30 together with the outer chamber wall. Preferably, the plurality of laterally-disposed inlet ports 38 communicating through the conduit wall 36 and opening into the channel 60 of the conduit 22 are adapted for conveying fluid medical gas from a fluid medical gas supply directly into a patient's mouth, the opening 28 in the mouthpiece 26, and the channel 60, thus being adapted to allow inhalation suction by the patient of the fluid medical gas, as well as exhalation of exhalant through the same channel 60, out the outlet valve and into the exhalant chamber 30. Exhalant is prevented from passing outwardly through the inlet ports 38 primarily by positive pressure gas flow from the medical gas supply, and this is reinforced, secondarily, by vacuum suction of exhalant through outlet ports 44.

In an embodiment, the mouthpiece 26, the conduit 22, and a portion of the exhalant chamber 30 comprising the dividing wall 52 (shown in lighter shading in FIGS. 2A-2C), as well as the support structure 53, which is preferable and optional, may all be integrally molded as a single unit. In this regard, the dividing wall 52 is preferably cup-shaped and molded partially around the mouthpiece 26 and around the conduit 22 such that a first end 64 of the conduit 22 interfaces integrally with a posterior portion of the mouthpiece and a second end 66 of the conduit interfaces integrally with an anterior-most portion of the cup-shaped dividing wall 52 defining at that location a valve opening in the dividing wall for communicating between the conduit 22 and the exhalant chamber, the inner cup-shaped dividing wall 52 partially defining an inner wall of the exhalant chamber 30.

Moreover, thus, preferably the exhalant chamber 30 (comprised of partially nested and cup-shaped inner dividing wall 52 and outer exhalant chamber wall 54, partially surrounds the preferably integral unit comprised of the conduit 26, the mouthpiece 26, and the dividing wall 52. In this preferred embodiment, the exhalant chamber 30 outer wall 54 preferably also comprises an integral unit defining a plurality of interconnection outlet tubes 68 communicating with the exhalant chamber 30 and defining the at least one outlet port 44, but preferably a plurality (e.g., two) outlet ports 44, adapted for fluid communication with, and interconnection to, the exhalant tubing 46 available in the surgery/procedure suite and connected to the vacuum system for safely evacuating the exhalant to an outside environment as known in the art. As such, the outer wall 54 narrows down into the plurality of tubes 68 communicating with the exhalant chamber 30. Outlet ports 44 are thus enabled, via tubes 68, in interconnecting with the evacuation tubes 46.

Thus, preferably, each of the tubes 34 and inlet ports 38 passes through outer wall 54 of the exhalant chamber 30 to open into the channel 60 formed by the conduit wall 36 allowing the flow of fluid medical gas from the fluid medical gas supply into the conduit. Thus, preferably, each of the plurality of outlet ports 44 communicates through the outer exhalant chamber wall 54 to open into the exhalant chamber 30 adapted for allowing evacuation of exhalant through the tubing under vacuum pressure. In this way, the inner exhalant chamber dividing wall 52 defines the plurality of preferably laterally-disposed inlet ports 38 adapted for communicating through the outer exhalant chamber wall 54, through the inner exhalant chamber wall 52, and through openings into the conduit wall 36 adapted for communicating with the channel 60 for exclusive oral delivery of medical gas to the patient.

Thus, preferably, in an embodiment of the disclosure, the apparatus 20 is comprised of an outer exhalant chamber wall 54 of integrally molded plastic, wherein the inner exhalant chamber wall 52, the conduit wall 36, the tubes 34, and the mouthpiece 26 are all integrally molded of plastic, it being the case that the inner support structure 53 is also optionally part of this integrated molded combination. Since the outer exhalant chamber wall 54 and the inner exhalant chamber wall 52 define outer and inner nested cups, respectively, and the outer exhalant wall 54 further comprises a plurality of ribbed spacers 56 for maintaining venting of the exhalant chamber 30 between the outer exhalant chamber wall 54 and the inner exhalant chamber wall 52, adequate flow of exhalant under vacuum pressure is assured. The inner exhalant chamber wall 52 also forms a lip 69, and since the inner exhalant chamber wall 52 is soft and adapted for comfortably engaging against the patient's face, the lip 69 is adapted for comfortably engaging the exterior of the patient's upper lip, lower lip, and sides of the patient's face in an annular fashion around the patient's mouth.

In an embodiment, the mouthpiece 26 further comprises an annular inner lip member 48 having laterally-disposed wings 49, all adapted for being inserted in the patient's mouth and sealing against inner portions of the patient's mouth just inside the patient's lips to facilitate holding the mouthpiece 26 in the patient's mouth and delivery of fluid medical gas into the patient's mouth. In such an embodiment the mouthpiece also preferably further comprises bite members 50 with integral teeth ledges 51 extending integrally from the mouthpiece inner lip member so as to be adapted for insertion between the patient's teeth and to facilitate the patient's ability to hold the mouthpiece 26 in the patient's mouth. Further, in a preferred embodiment, the mouthpiece 26 is preferably integrated with, and partially defines, the conduit 22, for facilitating delivery of the fluid medical gas into the patient's mouth through the inlet ports 38, into the conduit 22, and out the opening 28 defined in the mouthpiece 26, as shown by medical gas flow arrows 1.

In an alternative embodiment in accordance with another aspect of the disclosure, there is provided an apparatus for oral delivery of fluid medical gas to a patient further comprising means 71, 73 adapted for occluding at the posterior aspect of the patient's nasal airway to prevent transmission of fluids into the oral cavity during performance of surgery or a procedure while using the apparatus.

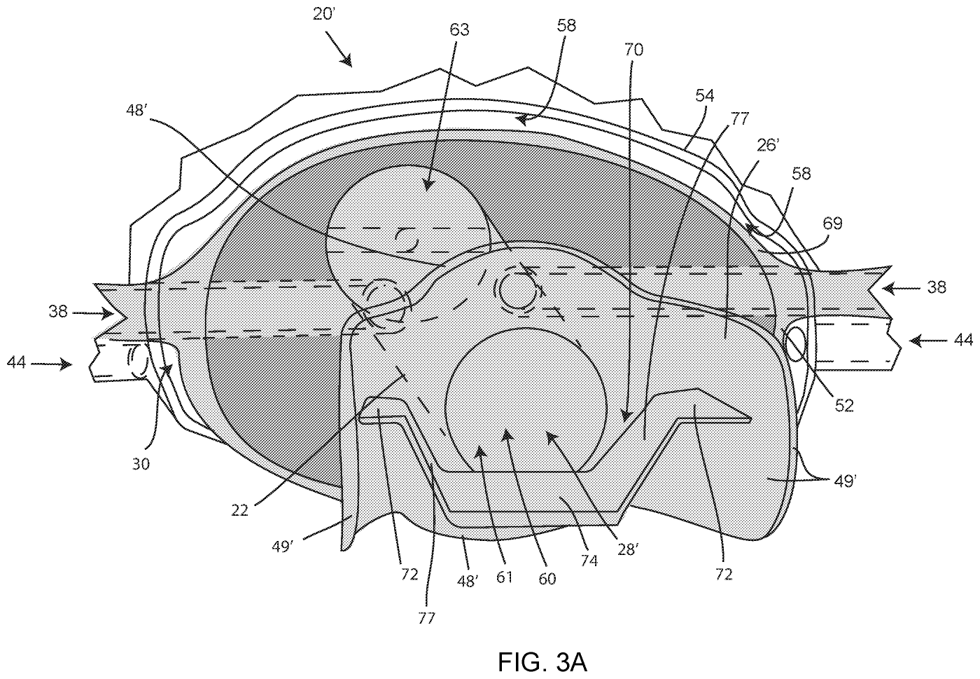

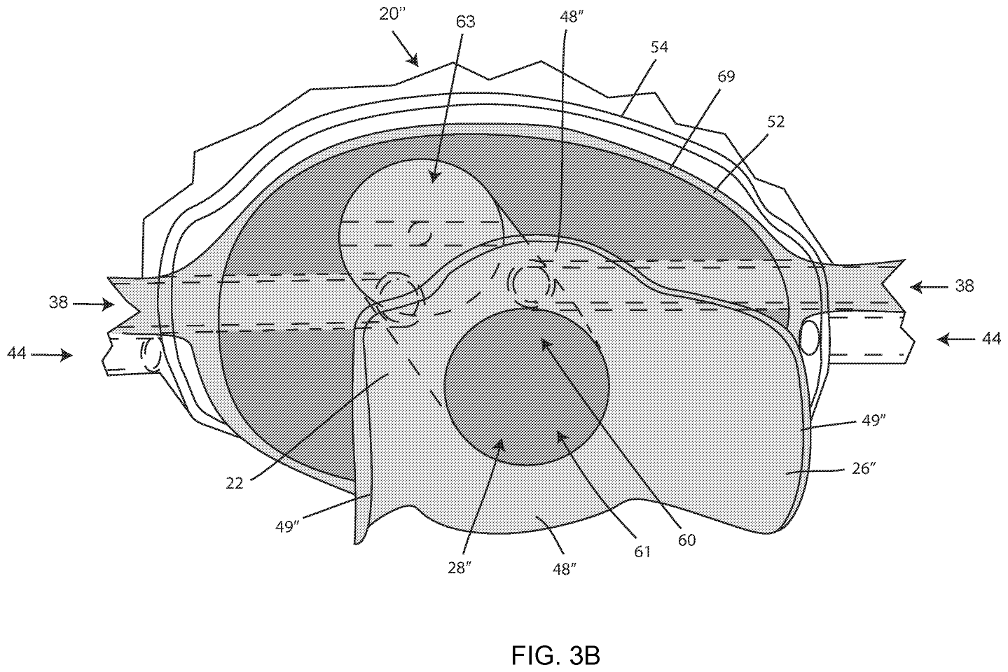

Referring now to FIGS. 3A-3B, there are provided a plurality of alternative embodiment mouth piece structures 26′, 26″, respectively. Thus, in accordance with one or more of the foregoing aspects of the disclosure, there is provided an alternative embodiment mouthpiece 26′ (FIG. 3A) wherein the mouthpiece 26′ of an apparatus 20′ further comprises a combination bite and tongue engagement member 70, further comprised of a bite area 72, tongue member support walls 77, and a tongue depressing area 74, adapted for insertion between the patient's teeth and engagement in depressing the patient's tongue to facilitate the patient's ability to hold the mouthpiece 26′ in the patient's mouth for delivery of the fluid medical gas into the patient's mouth.

Thus, in accordance with this alternate embodiment, there is provided an apparatus 20 for oral delivery of fluid medical gases to a patient, comprising: a molded inner mouthpiece 26′ comprising a annular inner lip member 48′ defining a mouthpiece opening 28′, a conduit wall 36 extending from the mouthpiece opening 28′ in the annular lip member 48′ of the mouthpiece 26′, the conduit wall defining a channel 60 having first and second end openings 61, 63, the first end opening 61 communicating with the mouthpiece opening 28′, and the second opening 63 communicating with the dividing wall opening 42 for holding the one-way outlet valve 43 in the dividing wall 52.

Further, in accordance with one or more of the foregoing aspects of the disclosure, there is further provided another alternative embodiment mouthpiece 26″ (FIG. 3B) wherein the mouthpiece 26″ of an apparatus 20″ further comprises a annular inner lip member 48″ to facilitate the patient's ability to hold the mouthpiece 26″ in the patient's mouth for delivery of the fluid medical gas into the patient's mouth.

Thus, in accordance with this other alternative embodiment, there is provided an apparatus 20 for oral delivery of fluid medical gases to a patient, comprising: a molded inner mouthpiece 26″ comprising a annular inner lip member 48″ defining a mouthpiece opening 28″, a conduit wall 36 extending from the mouthpiece opening 28″ in the annular lip member 48″ of the mouthpiece 26″, the conduit wall defining a channel 60 having first and second end openings 61, 63, the first end opening 61 communicating with the mouthpiece opening 28″, and the second opening 63 communicating with the dividing wall opening 42 for holding a one-way outlet valve 43 in the dividing wall 52.

While a preferred embodiment of the present invention has been shown and described, it will be apparent to those skilled in the art that many changes and modifications may be made without departing from the invention in its broader aspects. The appended claims are therefore intended to cover all such changes and modifications as fall within the true scope of the invention.

What is claimed is:

1. An apparatus for direct oral delivery of fluid medical gas to a patient, comprising:
a conduit adapted for fluid communication with fluid medical gas supply for introduction of the fluid medical gas into the patient's mouth;
a mouthpiece in fluid communication with said conduit and adapted for facilitating the introduction of fluid medical gas directly into the patient's mouth;
an exhalant chamber at least partially divided from said conduit;
an outlet valve in one-way fluid communication between said conduit and said exhalant chamber, said outlet valve adapted for permitting exhalation of combined breath, exhaled fluid medical gas, and any saliva, as exhalant by the patient, into said exhalant chamber, said outlet valve preventing inhalation of any fluid gas present in said exhalant chamber, wherein said exhalant chamber is adapted for porting of the exhalant outside of an environment in which the apparatus is adapted for use.

2. The apparatus of claim 1, wherein said mouthpiece further comprises an outer lip adapted for sealing against the patient's lips to facilitate delivery of fluid medical gas into the patient's mouth.

3. The apparatus of claim 1, wherein said mouthpiece further comprises a bite member adapted for insertion between the patient's teeth to facilitate the patient's ability to hold said mouthpiece in the patient's mouth for delivery of the fluid medical gas into the patient's mouth.

4. The apparatus of claim 1, further comprising a combination bite member and palate engagement member adapted for insertion between the patient's teeth and engagement with the patient's tongue to facilitate the patient's ability to hold said mouthpiece in the patient's mouth for delivery of the fluid medical gas into the patient's mouth.

5. The apparatus of claim 1, wherein said mouthpiece is integral said conduit for facilitating delivery of the fluid medical gas into the patient's mouth.

6. The apparatus of claim 1, further comprising an inlet port adapted for fluid communication between said conduit and the fluid medical gas supply for delivery of the fluid medical gas into the patient's mouth.

7. The apparatus of claim 6, further comprising another inlet port for communication between said conduit and the fluid medical gas supply for delivery of the fluid medical gas into the patient's mouth.

8. The apparatus of claim 1, further comprising a dividing wall, wherein said conduit is defined integral with mouthpiece, wherein said exhalant chamber further comprises an outer chamber wall anterior of said conduit and mouthpiece, said exhalant chamber further comprising a vented cavity or chamber between the outer chamber wall and said conduit and mouthpiece, and wherein said conduit and mouthpiece are at least partially divided from said exhalant chamber by said dividing wall.

9. The apparatus of claim 1, wherein said exhalant chamber further comprises at least one outlet port adapted for fluid communication with tubing connected to a vacuum system for evacuating the exhalant.

10. The apparatus of claim 1, further comprising means adapted for occluding at the rear of the posterior aspect of a patient's nasal cavity toward the nasopharyngeal junction to prevent transmission of fluids into the patient's oral cavity during performance of surgery or a procedure.

11. The apparatus of claim 1, wherein said conduit, said mouthpiece, and said dividing wall are an integral unit; and wherein said exhalant chamber partially surrounds the integral unit comprised of said conduit, said mouthpiece, and said dividing wall, said exhalant chamber further comprising at least one outlet port adapted for fluid communication with tubing connected to a vacuum system for evacuating the exhalant.

12. An apparatus for oral delivery of fluid medical gas to a patient, comprising:
a mouthpiece comprising a lip defining an opening;
a conduit interconnected with said mouthpiece and having a channel opening aligned with the opening defined in the lip of said mouthpiece adapted for fluid communication with fluid medical gas supply for introduction of the fluid medical gas directly into and through the patient's mouth through said conduit and the opening in said mouthpiece, said conduit defining at least one inlet port adapted for fluid communication between the mouthpiece, the conduit, and fluid medical gas supply;
an inner exhalant chamber wall molded at least partially around said mouthpiece so that fluid medical gas passing through the conduit and the opening in the lip of said mouthpiece is prevented from flowing directly into the exhalant chamber, said exhalant chamber further comprising at least one outlet port adapted for fluid communication with tubing connected to a vacuum system for evacuating the exhalant;
an outlet valve in one-way fluid communication between said conduit and said exhalant chamber, said outlet valve adapted for permitting exhalation of combined breath, exhaled fluid medical gas, and any saliva, as exhalant by the patient through said conduit and into said exhalant chamber, said outlet valve preventing inhalation of any fluid gas present in said exhalant chamber, wherein said exhalant chamber is adapted for porting of the exhalant outside of an environment in which the apparatus is adapted for use.

13. The apparatus of claim 12, wherein said at least one inlet port comprises a plurality of inlet ports adapted for fluid communication with the fluid medical gas supply, and wherein said at least one outlet port comprises a plurality of outlet ports adapted for fluid communication with tubing connected to the vacuum system for evacuating the exhalant.

14. The apparatus of claim 12, wherein said mouthpiece further comprises an outer lip adapted for sealing against the patient's lips to facilitate delivery of fluid medical gas into the patient's mouth.

15. The apparatus of claim 14, wherein said mouthpiece further comprises a bite member adapted for insertion between the patient's teeth to facilitate the patient's ability to hold said mouthpiece in the patient's mouth for delivery of the fluid medical gas into the patient's mouth.

16. The apparatus of claim 12, further comprising a combination bite member and palate engagement member adapted for insertion between the patient's teeth and engagement with the patient's palate to facilitate the patient's ability to hold said mouthpiece in the patient's mouth for delivery of the fluid medical gas into the patient's mouth.

17. The apparatus of claim 13, wherein each of the plurality of inlet ports communicates in sealed fashion through said exhalant chamber and said mouthpiece and opens into the conduit adapted for allowing flow of fluid medical gas from the fluid medical gas supply into the conduit, and wherein each of the plurality of outlet ports communicate through the exhalant chamber wall to open into the exhalant chamber adapted for allowing evacuation of exhalant through the tubing under vacuum pressure.

18. An apparatus for oral delivery of fluid medical gases to a patient, comprising:
a molded inner mouthpiece comprising a annular lip defining an intermediate opening;
a conduit wall extending from the intermediate opening in the annular lip of said mouthpiece, said conduit wall defining a channel having first and second end openings, the first end opening communicating with the intermediate opening in said mouthpiece;
a plurality of laterally-disposed inlet ports communicating through said conduit wall and opening into the channel adapted for conveying fluid medical gas from a fluid medical gas supply directly into a patient's mouth, the opening in said mouthpiece and said channel adapted to allow inhalation suction by the patient of the fluid medical gas;
an intermediate inner exhalant chamber wall molded partially around said mouthpiece and said conduit wall, said inner exhalant chamber wall defining an opening therein communicating with the second opening of said conduit wall;
an outer exhalant chamber wall molded partially around said inner exhalant chamber wall and defining a vented exhalant chamber between said outer exhalant chamber wall and said inner exhalant chamber wall, said outer exhalant chamber wall further defining a plurality of laterally-disposed outlet ports adapted for communicating through said outer exhalant chamber wall and thus between the exhalant chamber and tubes of a vacuum system for evacuating exhalant outside of the environment in which the apparatus is to be used, aid inner exhalant chamber wall defining a plurality of laterally-disposed inlet ports adapted from communicating through said outer exhalant chamber wall, through said inner exhalant chamber wall, and through and opening into said conduit wall adapted for communicating with the channel for exclusive oral delivery of medical gas to the patient;
a positive-pressure outlet valve in one-way fluid communication between the channel defined in said conduit wall and the vented exhalant chamber, said outlet valve residing in the opening defined in said inner exhalant chamber wall and adapted for permitting exhalation of combined breath, exhaled fluid medical gas, and any saliva, as exhalant by the patient, into the vented exhalant chamber, said outlet valve preventing inhalation of any fluid gas or saliva present in the vented exhalant chamber, and wherein the vented exhalant chamber is adapted for porting of the exhalant through the plurality of outlet ports to outside of an environment in which the apparatus is adapted for use.

19. The apparatus of claim 18, wherein said outer exhalant chamber wall is integrally molded of plastic, wherein said inner exhalant chamber wall, said conduit wall, and said mouthpiece are integrally molded of plastic, and wherein said outer exhalant chamber wall and said inner exhalant chamber wall define outer and inner nested cups, said outer exhalant wall further comprising ribbed spacers for maintaining venting of the exhalant chamber between said outer exhalant chamber wall and said inner exhalant chamber wall.

20. The apparatus of claim 19, wherein the laterally-disposed inlet ports are comprised of a plurality of tubes passing through said outer exhalant chamber wall, said inner exhalant chamber wall, and through and opening into said conduit wall, the plurality of tubes adapted for interconnecting with corresponding medical gas supply tubes from a medical gas supply cannister, and wherein the laterally-disposed outlet ports are comprised of another plurality of tubes passing through said outer exhalant chamber wall and opening into said exhalant chamber, the another plurality of tubes adapted for interconnecting with corresponding evacuation tubes for scavenging exhalant, together with ambient air through the venting for the exhalant chamber, to the area outside the environment in which the apparatus is to be used.
